(19) United States
(12) Patent Application Publication
(10) Pub. No.: US 2024/0374872 A1
(43) Pub. Date: Nov. 14, 2024

(54) GUIDEWIRE DEVICE

(71) Applicant: ABIOMED, Inc., Danvers, MA (US)

(72) Inventor: Christopher KORKUCH, Danvers, MA (US)

(73) Assignee: ABIOMED, Inc., Danvers, MA (US)

(21) Appl. No.: 18/657,926

(22) Filed: May 8, 2024

Related U.S. Application Data
(60) Provisional application No. 63/464,806, filed on May 8, 2023.

(51) Int. Cl. A61M 25/09

(52) U.S. Cl. CPC A61M 25/09; A61M 2025/09008; A61M 2025/09175; A61M 2205/0266

(57) ABSTRACT

A guidewire device, including a system, kit, and method of use, are disclosed. The guidewire device may include a proximal guidewire portion operably coupled to a proximal joint, which is operably coupled to an expandable portion, which is operably coupled to a distal joint, which is operably coupled to a distal tip portion. The proximal guidewire portion may include a first flexible sidewall defining a first central lumen and may include a tubular member defining a pressure lumen. The proximal joint may include a proximal hypotube. The expandable portion may include an expandable sidewall defining a second central lumen and may be fluidly coupled to the pressure lumen. The expandable portion may have a deflated configuration and an inflated configuration. The distal joint may include a distal hypotube. The distal tip portion may include a second flexible sidewall defining a third central lumen.

### CROSS-REFERENCE TO RELATED APPLICATIONS

The present application claims priority to U.S. Provisional Patent Application No. 63/464,806, filed May 8, 2023, the contents of which are incorporated by reference herein in its entirety.

### TECHNICAL FIELD

The present disclosure is drawn to guidewire devices, and particularly guidewire devices capable of occluding a blood vessel.

### BACKGROUND

In some medical treatments, access to blood vessels (such as the iliac or femoral artery) is required through an access point, and this is conventionally done using large-bore access devices. To date, closure of such access points remains problematic. Conventional closure techniques still provide significant risk of vascular access complications and bleeding. Techniques involving a second access site (typically, contralateral access) may be used for bailout of potential complications, but the additional procedural time, contrast, radiation, etc., limit the effectiveness of such techniques. Further, in some scenarios, a second access site may not be available.

### BRIEF SUMMARY

In some embodiments, a guidewire device may be provided. The guidewire device may include a proximal guidewire portion, a proximal joint, an expandable portion, a distal joint, and a distal tip portion. The proximal guidewire portion may include a first flexible sidewall defining a first central lumen extending therethrough. The proximal guidewire portion may include a tubular member defining a pressure lumen extending therethrough. The proximal joint may include a proximal hypotube. A proximal end of the proximal hypotube may be coupled to a distal end of the proximal guidewire portion. The pressure lumen may extend at least partially into the proximal hypotube. The expandable portion may include an expandable sidewall defining a second central lumen therethrough. A proximal end of the expandable portion may be coupled to a distal end of the proximal hypotube and fluidly coupled to the pressure lumen. The expandable portion may be movable between a deflated configuration in which the expandable portion has a first outer diameter and an inflated configuration in which the expandable portion has a second outer diameter greater than the first outer diameter. The distal joint may include a distal hypotube. A proximal end of the distal hypotube may be coupled to a distal end of the expandable portion. The distal tip portion may include a second flexible sidewall defining a third central lumen extending therethrough. The distal tip portion may be coupled to a distal end of the distal hypotube.

In some embodiments, an outer surface of the proximal end of the expandable portion may be adhered to an inner surface of a distal end of the proximal hypotube and a distal end of the pressure lumen may be adhered to an inner surface of a proximal end of the proximal hypotube.

In some embodiments, an outer surface of the distal end of the expandable portion may be coupled to an inner surface of a proximal end of the distal hypotube. In some embodiments, an adhesive within the distal hypotube may fill a sufficient volume to allow an inflation fluid entering the expandable portion to pressurize the expandable portion.

In some embodiments, the proximal guidewire portion, proximal joint portion, distal joint portion, and distal tip portion may have a constant outer diameter substantially equal to the first outer diameter of the expandable portion. In some embodiments, the constant outer diameter is about 0.035 inches.

In some embodiments, the guidewire device may include a stiffening wire extending through at least the first central lumen and a portion of the second central lumen. In some embodiments, the guidewire device may include a distal tip wire extending through at least a portion of the third central lumen. The distal tip wire may have an outer diameter that is greater than an outer diameter of the stiffening wire. The distal tip wire may be coupled to the stiffening wire within the distal hypotube. In some embodiments, a flexible distal tip may be coupled to a distal end of the distal tip wire. In some embodiments, the flexible distal tip may be a straight tip or a J-tip.

In some embodiments, the first flexible sidewall and the second flexible sidewall may each independently include nitinol or stainless steel. In some embodiments, the proximal hypotube and the distal hypotube may each independently include nitinol or stainless steel. In some embodiments, the tubular member defining the pressure lumen may include polyetheretherketone (PEEK), a polyether block amide (PEBA), acrylonitrile butadiene styrene (ABS), polypropylene, polyethylene, polytetrafluoroethylene (PTFE), fluorinated ethylene propylene (FEP), a perfluoroalkoxy alkane (PFA), a nylon, a polyimide, and/or a liquid crystal polymer (LCP). In some embodiments, the expandable sidewall may include a thermoplastic polyurethane (TPU), a styrenic block copolymer, a natural or synthetic rubber, polyisoprene, and/or ethylene-vinyl acetate (EVA). In some embodiments, the guidewire device may include a radiopaque material.

In some embodiments, the expandable portion may be configured to occlude a vessel of up to 13 mm in diameter when the expandable portion is in the inflated configuration.

In some embodiments, the guidewire device may include a coupling at a proximal end of the guidewire device. The coupling may be configured to removably attach an inflation device to the guidewire device. The coupling may be configured to fluidically connect the inflation device to the pressure lumen.

In some embodiments, a system may be provided. The system may include an embodiment of a guidewire device as disclosed herein. The system may include an inflation device that may be removably attached to a proximal end of the guidewire device.

In some embodiments, a kit may be provided. The kit may include an embodiment of a guidewire device as disclosed herein. The kit may include an inflation device that may be removably attached to a proximal end of the guidewire device. In some embodiments, the kit may include an introducer sheath which may have a lumen therethrough adapted to slidably receive the guidewire device. In some embodiments, the kit may include a closure device, such as an arterial closure device, which may have a lumen adapted to slidably receive the guidewire device.

In some embodiments, a method may be provided. The method may include inserting an embodiment of a guidewire device as disclosed herein into a blood vessel of a subject via a lumen of an access device. The method may include attaching an inflation device to a proximal end of the guidewire device and inflating the expandable portion to a desired degree of occlusion of the blood vessel. The method may include detaching the inflation device from the guidewire device while maintaining a pressure and degree of occlusion. The method may include removing the access device over the guidewire device. The method may include performing a therapeutic procedure. The method may include reattaching the inflation device to the proximal end of the guidewire device and deflating the expandable portion. The method may include removing the guidewire device from the subject. The method may include verifying a desired level of hemostasis is achieved after removing the access device, prior to performing the therapeutic procedure. The method may include visualizing hemostasis is achieved at the access site after deflating the expandable portion and prior to removal of the guidewire device.

In some embodiments, performing a therapeutic procedure may include positioning an arterial closure device along the guidewire device, the arterial closure device including a lumen configured to slidably receive the guidewire device.

### BRIEF DESCRIPTION OF DRAWINGS

The accompanying drawings, which are incorporated in and constitute a part of this specification, illustrate embodiments of the present invention and, together with a general description of the invention given above, and the detailed description of the embodiments given below, serve to explain the principles of the present invention.

FIG. 1 is an illustration of a system utilizing a guidewire device.

FIG. 2 is a cross-sectional illustration of a portion of a guidewire device.

FIG. 3A is an illustration of a guidewire device interacting with an access device.

FIG. 3B is an illustration of a guidewire device interacting with a closure device.

FIG. 4 is a flowchart of a method.

It should be understood that the appended drawings are not necessarily to scale, presenting a somewhat simplified representation of various features illustrative of the basic principles of the invention. The specific design features of the sequence of operations as disclosed herein, including, for example, specific dimensions, orientations, locations, and shapes of various illustrated components, will be determined in part by the particular intended application and use environment. Certain features of the illustrated embodiments have been enlarged or distorted relative to others to facilitate visualization and clear understanding. In particular, thin features may be thickened, for example, for clarity or illustration.

### DETAILED DESCRIPTION

The present disclosure relates to devices, systems, and methods that allows the closure or blockage of a blood vessel located, e.g., proximally to an access site, through a single access site, while also allowing for the use of conventional access devices while the closure or blockage is occurring.

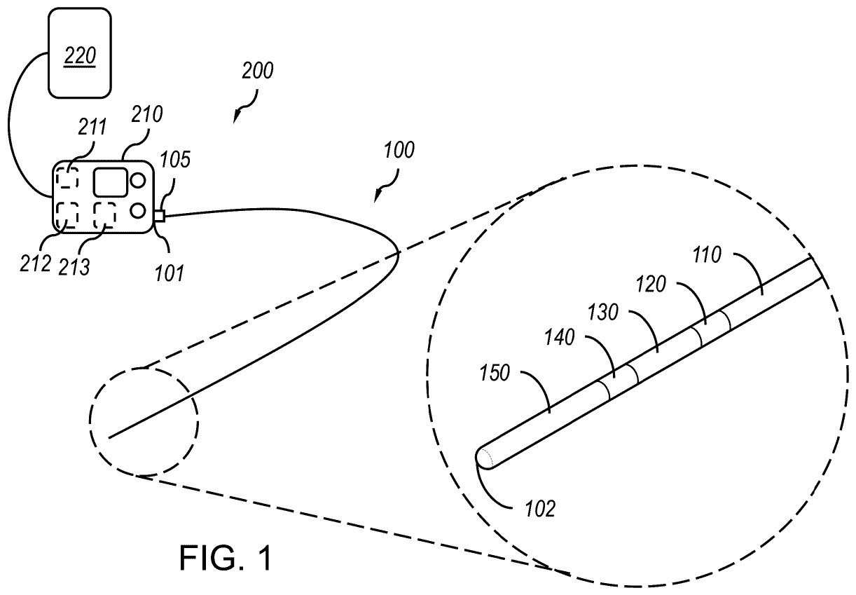

Referring to FIG. 1, a system can be seen. The system (200) may include an embodiment of a guidewire device (100) as disclosed herein attached to an inflation device (210). The guidewire device may have a proximal end (101) and a distal end (102). The guidewire device (100) may include a coupling (105) at proximal end (101) of the guidewire device. The coupling may be configured to, e.g., removably attach the inflation device to the guidewire device. The guidewire device (100) may include a proximal guidewire portion (110), a proximal joint (120), an expandable portion (130), a distal joint (140), and a distal tip portion (150).

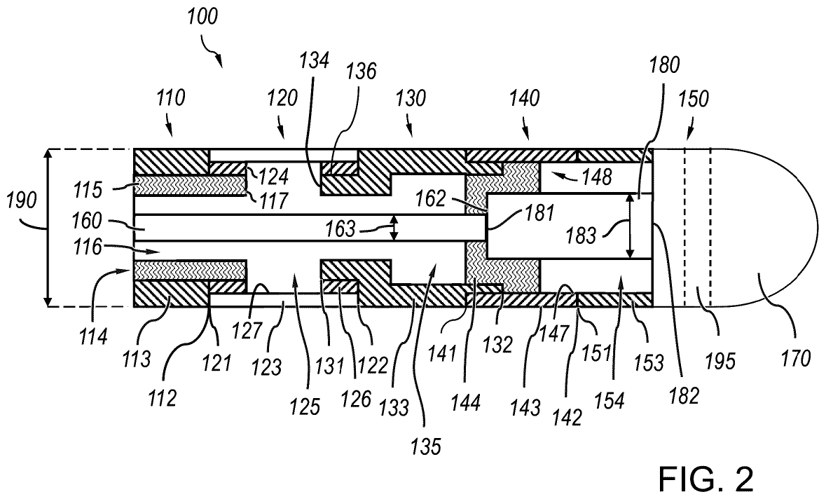

Referring to FIG. 2, a portion of a guidewire can be seen.

The proximal guidewire portion (110) may include a first flexible sidewall (113) defining a first central lumen (114) extending therethrough. In some embodiments, the first flexible sidewall (113) may include nitinol or stainless steel.

The proximal guidewire portion may include a tubular member (115) defining a pressure lumen (116) extending therethrough. In some embodiments, the tubular member (115) may be in contact with the first flexible sidewall (113). In some embodiments, the tubular member (115) may extend through the first central lumen (114).

In some embodiments, the tubular member defining the pressure lumen may include polyetheretherketone (PEEK), a polyether block amide (PEBA), acrylonitrile butadiene styrene (ABS), polypropylene, polyethylene, polytetrafluoroethylene (PTFE), fluorinated ethylene propylene (FEP), a perfluoroalkoxy alkane (PFA), a nylon, a polyimide, and/or a liquid crystal polymer (LCP).

Referring briefly to both FIGS. 1 and 2, in some embodiments, the coupling (105) may be configured to fluidically connect the inflation device (210) to the pressure lumen (116).

Referring to FIG. 2, the proximal joint (120) may include a proximal hypotube (123). A proximal end (121) of the proximal hypotube (123) may be coupled to a distal end (112) of the proximal guidewire portion (110). In some embodiments, the proximal hypotube include nitinol or stainless steel.

In some embodiments, the pressure lumen (116) may extend at least partially into the proximal hypotube (123). At least part of the pressure lumen (116) may be within a lumen 125 defined by the proximal hypotube (123). In some embodiments, the tubular member may be coupled to the proximal hypotube.

In some embodiments, an adhesive (124) may be used to couple the tubular member to the proximal hypotube. In some embodiments, the adhesive (124) may be in contact with the first flexible sidewall (113). In some embodiments, the distal end (117) of the tubular member (115) may be adhered (e.g., via adhesive (124)) to an inner surface (127) of a proximal end (121) of the proximal hypotube (123).

The expandable portion (130) may include an expandable sidewall (133) defining a second central lumen (135) therethrough.

In some embodiments, the expandable sidewall may include a thermoplastic polyurethane (TPU), a styrenic block copolymer, a natural or synthetic rubber, polyisoprene, and/or ethylene-vinyl acetate (EVA). In some embodiments, the expandable sidewall may comprise a plurality of materials, including, e.g., one or more polymers and one or more metals.

A proximal end (131) of the expandable portion (130) may be coupled to a distal end 122 of the proximal hypotube (123). The proximal end may be fluidly coupled to the pressure lumen (116). In some embodiments, a distal end (117) of tubular member (115) is separated from a proximal end (134) of the expandable sidewall (133). In some embodiments, the distal end of tubular member is in contact with the proximal end of the expandable sidewall.

In some embodiments, the expandable portion may be configured to occlude a vessel of up to 13 mm in diameter when the expandable portion is in the inflated configuration. In some embodiments, the expandable portion may be configured to occlude a vessel from 1 mm, 2 mm, 3 mm, 4 mm, or 5 mm in diameter up to 10 mm, 11 mm, 12 mm, or 13 mm in diameter when the expandable portion is in the inflated configuration, including all combinations and subranges thereof.

The outer diameter (190) of the expandable portion may be controllable. In some embodiments, the expandable portion may be movable between a deflated configuration in which the expandable portion has a first outer diameter and an inflated configuration in which the expandable portion has a second outer diameter greater than the first outer diameter.

In some embodiments, the change from deflated to inflated configuration may be accomplished in one or more ways. In some embodiments, the change from deflated to inflated configuration may be accomplished by providing a fluid to the expandable portion at a predetermined pressure. In some embodiments, the second outer diameter may vary based on, e.g., the vessel geometry, properties of the vessel, properties of the expandable portion, etc. In some embodiments, the change from deflated to inflated configuration may be accomplished by providing a predetermined volume of a fluid to the expandable portion. In some embodiments, the second outer diameter may be determined based on, e.g., the geometry of the guidewire device. In some embodiments, the expandable portion may be inflated to a desired second diameter. In some embodiments, the expandable portion may include a radiopaque material, and the second outer diameter may be monitored during inflation until the desired second outer diameter is reached. In some embodiments, a volume of fluid (and/or a pressure of the fluid) necessary to achieve a desired second outer diameter is calculated, and the appropriate volume and/or pressure of the fluid is then provided to the expandable portion.

In some embodiments, an outer surface (136) of the proximal end (131) of the expandable portion (130) may be coupled to an inner surface (127) of a distal end 122 of the proximal hypotube (123). In some embodiments, an outer surface (136) of the proximal end (131) of the expandable portion (130) may be adhered (e.g., via an adhesive (126)) to an inner surface (127) of a distal end 122 of the proximal hypotube (123).

The distal joint (140) may include a distal hypotube (143). In some embodiments, the distal hypotube may include nitinol or stainless steel. In some embodiments, the proximal and distal hypotubes may include identical materials. In some embodiments, the proximal and distal hypotubes may include different materials.

A proximal end (141) of the distal hypotube (143) may be coupled to a distal end (132) of the expandable portion (130).

In some embodiments, an outer surface (136) of the distal end (132) of the expandable portion (130) may be coupled to an inner surface (147) of a proximal end (141) of the distal hypotube (143).

In some embodiments, an adhesive (144) within the distal hypotube (143) (e.g., within a lumen (148) defined by the inner surface (147) of the distal hypotube (143)) may fill a sufficient volume to allow an inflation fluid (e.g., a fluid from an inflation device that passes through the pressure lumen) entering the expandable portion to pressurize the expandable portion. In some embodiments, the adhesive within the distal hypotube may be configured to prevent the inflation fluid from moving distally beyond the distal end (132) of the expandable portion (130).

The distal tip portion (150) may include a second flexible sidewall (153) defining a third central lumen (154) extending therethrough. The distal tip portion (150) may be coupled to a distal end (142) of the distal hypotube (143) (for example, proximal end (151) may be coupled to distal end (142) of the distal hypotube).

In some embodiments, the second flexible sidewall may include nitinol or stainless steel. In some embodiments, the first flexible sidewall and the second flexible sidewall may both include identical materials. In some embodiments, the first flexible sidewall and the second flexible sidewall may include different identical materials.

In some embodiments, the proximal guidewire portion, proximal joint portion, distal joint portion, and distal tip portion may have a constant outer diameter substantially equal to the first outer diameter of the expandable portion (e.g., the outer diameter (190) of the expandable portion when in the deflated configuration). As used herein, the term “substantially equal” refers to values that are within 20%, such as within 10%, such as within 5% of a target value. In some embodiments, the proximal guidewire portion, proximal joint portion, distal joint portion, and distal tip portion may have a maximum outer diameter substantially equal to the first outer diameter of the expandable portion. In some embodiments, the constant outer diameter is about 0.035 inches. As used herein, the term “about [a value]” refers to values within 5% of the value, such as within 2.5% of the value, such as within 1% of the value. In some embodiments, the first outer diameter is less than the constant outer diameter. In some embodiments, the maximum outer diameter is about 0.035 inches.

In some embodiments, the guidewire device (100) may include a stiffening wire (160) extending through at least the first central lumen (114) and a portion of the second central lumen (135). In some embodiments, the stiffening wire may extend through at least a portion of the lumen (148) defined by the distal hypotube (143). In some embodiments, the stiffening wire may extend through at least a portion of the third central lumen (154). A distal end (162) of the stiffening wire (160) may be disposed within an adhesive (144).

In some embodiments, the guidewire device (100) may include a distal tip wire (180) extending through at least a portion of the third central lumen (154). In some embodiments, the distal tip wire (180) may extend through at least a portion of the lumen (148) defined by the distal hypotube (143). In some embodiments, the distal tip wire (180) may extend through at least a portion of the second central lumen (135).

In some embodiments, the distal tip wire (180) may be coupled to the stiffening wire (160). For example, proximal end (181) of the distal tip wire (180) may be coupled to a distal end (162) of the stiffening wire (160). In some embodiments, the distal tip wire may be coupled to the stiffening wire within the expandable portion (130). In some embodiments, the distal tip wire may be coupled to the stiffening wire within the distal hypotube (143).

In some embodiments, the distal tip wire may have an outer diameter (183) that is greater than an outer diameter (163) of the stiffening wire. In some embodiments, the distal tip wire may have a maximum outer diameter that is greater than a maximum outer diameter of the stiffening wire. In some embodiments, the distal tip wire may have a minimum outer diameter that is less than a minimum outer diameter of the stiffening wire. In some embodiments, the distal tip wire may have a minimum outer diameter that is less than a minimum outer diameter of the stiffening wire.

In some embodiments, a flexible distal tip (170) may be coupled to a distal end (182) of the distal tip wire (180). In some embodiments, the flexible distal tip may be a straight tip or a J-tip.

In some embodiments, the guidewire device may include a radiopaque material. In some embodiments, a portion (195) of the flexible distal tip may include a radiopaque material. In some embodiments, the expandable portion may include a radiopaque material. In some embodiments, the proximal and distal junctions may include a radiopaque material.

In some embodiments, a kit may be provided. Referring to FIG. 1, the kit may include an embodiment of a guidewire device (100) as disclosed herein. The kit may include an inflation device (210) that may be removably attached to a proximal end of the guidewire device. The inflation device may include, e.g., one or more processing units (211) operably coupled to a non-transitory computer readable storage device (212).

As used herein, the term “processing unit” may include any combination of hardware, firmware, and software, employed to process data or digital signals. Processing unit hardware may include, for example, application specific integrated circuits (ASICs), general purpose or special purpose central processing units (CPUs), digital signal processors (DSPs), graphics processing units (GPUs), and programmable logic devices such as field programmable gate arrays (FPGAs). In a processor, as used herein, each function may be performed either by hardware configured, i.e., hard-wired, to perform that function, or by more general-purpose hardware, such as a CPU, configured to execute instructions stored in a non-transitory storage medium. A processor may be fabricated on a single printed wiring board (PWB) or distributed over several interconnected PWBs. A processor may contain multiple processing units; for example, a processor may include two processing units, an FPGA and a CPU, interconnected on a PWB.

The storage device may contain instructions that, when executed by the one or more processing units, control, e.g., operation of the inflation device.

The processing unit(s) may be operably coupled to a pumping device (213). The pumping device (213) may be any device configured to provide a fluid to the expandable portion (130) of the guidewire device (100). The pumping device (213) may be operably coupled to a fluid source (220) containing the fluid used to control the inflation of the expandable portion. The fluid may be, e.g., a gas or liquid. In some embodiments, the fluid may be a saline fluid. In some embodiments, the fluid source may be external to the pumping device. In some embodiments, the fluid source may be a portion of the pump (e.g., if the pump device includes a syringe, the fluid source may be the barrel of the syringe).

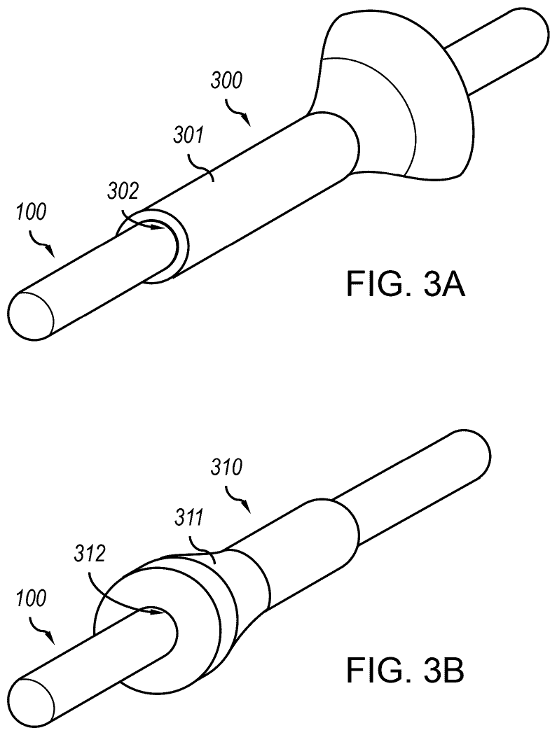

Referring to FIG. 3A, in some embodiments, the kit may include an introducer sheath (300). The introduced sheath may have a sheath body (301) that may define a lumen (302) extending therethrough. The lumen (302) may be adapted to slidably receive the guidewire device (100). Referring to FIG. 3B, in some embodiments, the kit may include a closure device (310), such as an arterial closure device. The closure device (310) may include a closure device body (311) that may define a lumen (312). The lumen (312) may be configured to slidably receive the guidewire device (100).

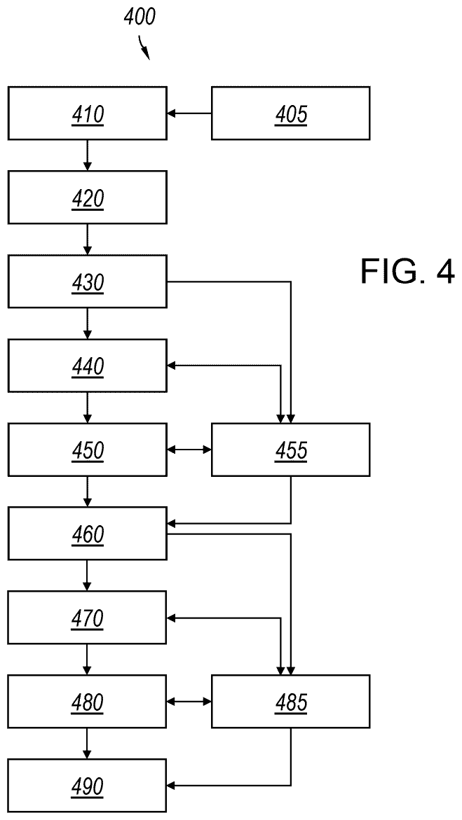

In some embodiments, a method may be provided. Referring to FIG. 4, the method (400) may include inserting (410) an embodiment of a guidewire device as disclosed herein into a blood vessel of a subject through an access point via a lumen of an access device. In some embodiments, the method may include positioning (405) the access device into the blood vessel prior to inserting the guidewire.

The method may include attaching (420) an inflation device to a proximal end of the guidewire device. This may be accomplished via, e.g., a coupling such as coupling (105).

The method may include inflating (430) the expandable portion to a desired degree of occlusion of the blood vessel. As described herein, this may include having the inflation device provide a fluid to the expandable portion, causing the expandable portion to move from a deflated configuration to an inflated configuration. In some embodiments, this may include providing the fluid at a predetermined pressure, providing a predetermined volume of fluid, and/or providing sufficient fluid to achieve a desired level of occlusion.

The method may include detaching (440) the inflation device from the guidewire device while maintaining a pressure and degree of occlusion.

The method may include removing (450) the access device over the guidewire device.

The method may include performing (460) a therapeutic procedure. The procedure may include involve one or more procedures that require the partial or complete blockage of at least one blood vessel. In some embodiments, performing a therapeutic procedure may include positioning an arterial closure device along the guidewire device, where the arterial closure device includes a lumen configured to slidably receive the guidewire device.

The method may include verifying (455) a desired level of hemostasis is achieved. In some embodiments, this step is performed once. In some embodiments, this step is performed multiple times. In some embodiments, this may be done after inflating (430) the expandable portion. In some embodiments, this may be done after detaching (440) the inflation device. In some embodiments, this may be done after removing (450) the access device. In some embodiments, it may be done prior to performing (460) the therapeutic procedure.

The method may include reattaching (470) the inflation device to the proximal end of the guidewire device.

The method may include deflating (480) the expandable portion. This may involve using the inflation device to remove some or all fluid from the guidewire, including from the expandable portion. In some embodiments, the inflation device is used to move the expandable portion from the inflated configuration to a deflated configuration.

The method may include removing (490) the guidewire device from the subject.

The method may include verifying (485) a desired level of hemostasis is achieved. This may include visualizing hemostasis is achieved at the access site. In some embodiments, this step is performed once. In some embodiments, this step is performed multiple times. This may be done performing (460) the therapeutic procedure. This may be done after reattaching (470) the inflation device. This may be done after deflating (480) the expandable portion. This may be done prior to removing (490) the guidewire device.

Embodiments of the present disclosure are described in detail with reference to the figures wherein like reference numerals identify similar or identical elements. It is to be understood that the disclosed embodiments are merely examples of the disclosure, which may be embodied in various forms. Well known functions or constructions are not described in detail to avoid obscuring the present disclosure in unnecessary detail. Therefore, specific structural and functional details disclosed herein are not to be interpreted as limiting, but merely as a basis for the claims and as a representative basis for teaching one skilled in the art.

Those skilled in the art will recognize or be able to ascertain using no more than routine experimentation many equivalents to the specific embodiments of the invention described herein.

Such equivalents are intended to be encompassed by the following claims.

1. A guidewire device, comprising:
a proximal guidewire portion comprising a first flexible sidewall defining a first central lumen extending therethrough, and further comprising a tubular member defining a pressure lumen extending therethrough;
a proximal joint comprising a proximal hypotube, a proximal end of the proximal hypotube coupled to a distal end of the proximal guidewire portion, the pressure lumen extending at least partially into the proximal hypotube;
an expandable portion comprising an expandable sidewall defining a second central lumen therethrough, a proximal end of the expandable portion coupled to a distal end of the proximal hypotube and fluidly coupled to the pressure lumen, wherein the expandable portion is movable between a deflated configuration in which the expandable portion has a first outer diameter and an inflated configuration in which the expandable portion has a second outer diameter greater than the first outer diameter;
a distal joint comprising a distal hypotube, a proximal end of the distal hypotube coupled to a distal end of the expandable portion; and
a distal tip portion comprising a second flexible sidewall defining a third central lumen extending therethrough, the distal tip portion coupled to a distal end of the distal hypotube.

2. The guidewire device of claim 1, wherein the proximal guidewire portion, proximal joint portion, distal joint portion, and distal tip portion have a constant outer diameter substantially equal to the first outer diameter of the expandable portion.

3. The guidewire device of claim 2, wherein the constant outer diameter is about 0.035 inches.

4. The guidewire device of claim 1, further comprising a stiffening wire extending through at least the first central lumen and a portion of the second central lumen.

5. The guidewire device of claim 4, further comprising a distal tip wire extending through at least a portion of the third central lumen.

6. The guidewire device of claim 5, wherein the distal tip wire has an outer diameter that is greater than an outer diameter of the stiffening wire.

7. The guidewire device of claim 5, wherein the distal tip wire is coupled to the stiffening wire within the distal hypotube.

8. The guidewire device of claim 5, wherein a flexible distal tip is coupled to a distal end of the distal tip wire.

9. The guidewire device of claim 8, wherein the flexible distal tip is a straight tip or a J-tip.

10. The guidewire device of claim 1, wherein the first flexible sidewall and the second flexible sidewall each independently comprises nitinol or stainless steel.

11. The guidewire device of claim 1, wherein the tubular member defining the pressure lumen comprises polyetheretherketone (PEEK), a polyether block amide (PEBA), acrylonitrile butadiene styrene (ABS), polypropylene, polyethylene, polytetrafluoroethylene (PTFE), fluorinated ethylene propylene (FEP), a perfluoroalkoxy alkane (PFA), a nylon, a polyimide, and/or a liquid crystal polymer (LCP).

12. The guidewire device of claim 1, wherein the proximal hypotube and the distal hypotube each independently comprise nitinol or stainless steel.

13. The guidewire device of claim 1, wherein the expandable sidewall is comprised of a thermoplastic polyurethane (TPU), a styrenic block copolymer, a natural or synthetic rubber, polyisoprene, and/or ethylene-vinyl acetate (EVA).

14. The guidewire device of claim 1, wherein the expandable portion is configured to occlude a vessel of up to 13 mm in diameter when the expandable portion is in the inflated configuration.

15. The guidewire device of claim 1, wherein an outer surface of the proximal end of the expandable portion is adhered to an inner surface of a distal end of the proximal hypotube and a distal end of the pressure lumen is adhered to an inner surface of a proximal end of the proximal hypotube.

16. The guidewire device of claim 1, wherein an outer surface of the distal end of the expandable portion is coupled to an inner surface of a proximal end of the distal hypotube, where an adhesive within the distal hypotube fills a sufficient volume to allow an inflation fluid entering the expandable portion to pressurize the expandable portion.

17. The guidewire device of claim 1, further comprising a coupling at a proximal end of the guidewire device, wherein the coupling is configured to removably attach an inflation device to the guidewire device and fluidically connect the inflation device to the pressure lumen.

18. The guidewire device of claim 1, wherein the guidewire device comprises a radiopaque material.

19. A system, comprising:
a guidewire device of claim 1; and
an inflation device removably attached to a proximal end of the guidewire device.

20. A kit, comprising:
a guidewire device of claim 1; and
an inflation device adapted to be removably attached to the guidewire device.

21-22. (canceled)

23. A method, comprising:
inserting a guidewire device into a blood vessel of a subject through an access point via a lumen of an access device, the guidewire device comprising:
a proximal guidewire portion comprising a first flexible sidewall defining a first central lumen extending therethrough, and further comprising a tubular member defining a pressure lumen extending therethrough;
a proximal joint comprising a proximal hypotube, a proximal end of the proximal hypotube coupled to a distal end of the proximal guidewire portion, the pressure lumen extending at least partially into the proximal hypotube;
an expandable portion comprising an expandable sidewall defining a second central lumen therethrough, a proximal end of the expandable portion coupled to a distal end of the proximal hypotube and fluidly coupled to the pressure lumen, wherein the expandable portion is movable between a deflated configuration in which the expandable portion has a first outer diameter and an inflated configuration in which the expandable portion has a second outer diameter greater than the first outer diameter;
a distal joint comprising a distal hypotube, a proximal end of the distal hypotube coupled to a distal end of the expandable portion; and
a distal tip portion comprising a second flexible sidewall defining a third central lumen extending therethrough, the distal tip portion coupled to a distal end of the distal hypotube;
attaching an inflation device to a proximal end of the guidewire device and inflating the expandable portion to a desired degree of occlusion of the blood vessel;
detaching the inflation device from the guidewire device while maintaining a pressure and degree of occlusion;
removing the access device over the guidewire device;
performing a therapeutic procedure;
reattaching the inflation device to the proximal end of the guidewire device and deflating the expandable portion; and
removing the guidewire device from the subject.

24-44. (canceled)
